(19) United States
(12) Patent Application Publication
(10) Pub. No.: US 2023/0046240 A1
(43) Pub. Date: Feb. 16, 2023

(54) AIRBAG, BLOOD PRESSURE MEASUREMENT APPARATUS, AND BLOOD PRESSURE MEASUREMENT METHOD

(71) Applicant: HUAWEI TECHNOLOGIES CO., LTD., Shenzhen (CN)

(72) Inventor: Yunsheng KUANG, Shenzhen (CN)

(73) Assignee: HUAWEI TECHNOLOGIES CO., LTD., Shenzhen (CN)

(21) Appl. No.: 17/785,190

(22) PCT Filed: Oct. 30, 2020
(86) PCT No.: PCT/CN2020/125381

§ 371 Date: Jun. 14, 2022

(30) Foreign Application Priority Data
Dec. 20, 2019 (CN) 201911324782.3

(51) Int. Cl. A61B 5/022; A61B 5/021; A61B 5/00

(52) U.S. Cl. CPC A61B 5/02241; A61B 5/02141; A61B 5/02108; A61B 5/681; A61B 2562/0247; A61B 2560/0257

(57) ABSTRACT

An airbag (10), a blood pressure measurement apparatus, and a blood pressure measurement method are provided. The blood pressure measurement apparatus includes a cavity (20), a pressing part (30), an air pump (40), a barometric pressure sensor (50), and an airbag (10). The airbag (10) is long-strip-shaped, the airbag (10) has a fixed end (113) and a free end (115), the fixed end (113) is fixed in the cavity (20), a plurality of grooves (101) are distributed on a surface of the airbag (10) along a length extension direction, and the grooves (101) extend along a width direction of the airbag (10). Each groove (101) can divide the airbag (10) into a pressurized area (110) and a non-pressurized area (115) along the length direction when the pressing part (30) movably disposed in the cavity (20) is pressed against the groove (101). The pressurized area (110) and the non-pressurized area (115) isolate air flow from each other, air holes (102) connecting the inside and the outside of the airbag (10) are distributed between every two adjacent grooves (101), and the cavity (20) is configured to accommodate the non-pressurized area (115) of the airbag (10), and when the airbag (10) does not work, accommodate the entire airbag (10). The blood pressure measurement apparatus in this application can adjust ratios of the pressurized area to wrist circumferences of different people. This improves blood pressure signal collection stability and blood pressure measurement accuracy.

This application claims priority to Chinese Patent Application No. 2019113247823, filed with the China National Intellectual Property Administration on Dec. 20, 2019 and entitled “AIRBAG, BLOOD PRESSURE MEASUREMENT APPARATUS, AND BLOOD PRESSURE MEASUREMENT METHOD”, which is incorporated herein by reference in its entirety.

### TECHNICAL FIELD

The embodiments relate to the field of blood pressure measurement technologies, an airbag, a blood pressure measurement apparatus, and a blood pressure measurement method.

### BACKGROUND

As living standard continuously improves, people pay more attention to their own health. Blood pressure, as a main physiological indicator of a human body, is also increasingly valued by people. Currently, product forms of sphygmomanometers on the market are mainly an upper-arm electronic sphygmomanometer and a wrist electronic sphygmomanometer. The two types of sphygmomanometers can perform single blood pressure measurement, but cannot perform dynamic blood pressure monitoring, and especially, blood pressure monitoring at night is difficult to perform. Therefore, miniaturization and wearableness of a sphygmomanometer become a trend, and a blood pressure watch emerges accordingly.

The blood pressure watch usually includes a watch core, a watch strap, an airbag, an air pump, and a barometric pressure sensor. Both the airbag and the watch strap may be sleeved on a wrist. Both the air pump and the barometric pressure sensor are disposed in the watch core. The air pump is configured to inflate the airbag. The barometric pressure sensor is configured to collect a pressure signal in the airbag to obtain a pulse wave signal, to implement blood pressure measurement.

However, a conventional blood pressure watch has different blood pressure measurement accuracy for different people. This is mainly reflected in different measurement accuracy for people with different wrist circumferences and causes difficulty to cover a wider range of users.

### SUMMARY

An objective of the embodiments is to provide an airbag, a blood pressure measurement apparatus, and a blood pressure measurement method, to resolve a problem that a blood pressure watch has different blood pressure measurement accuracy for people with different wrist circumferences.

To achieve the foregoing objective, the solutions used in the embodiments are as follows:

According to a first aspect, an embodiment provides an airbag, configured to measure blood pressure. The airbag is long-strip-shaped, a plurality of grooves is distributed on a surface of the airbag along a length extension direction of the airbag, the grooves extend along a width direction of the airbag, and each of the grooves is configured to divide the airbag into a pressurized area and a non-pressurized area along the length direction of the airbag when an external structure is pressed against the groove. The pressurized area and the non-pressurized area isolate air flow from each other, air holes connecting the inside and the outside of the airbag are distributed between every two adjacent grooves and blocking parts for blocking the air holes are further disposed in the airbag.

According to the airbag provided in this embodiment, the plurality of grooves is distributed in the length direction of the airbag, and each groove 101 is configured to divide the airbag into the pressurized area and the non-pressurized area along the length direction of the groove 101 when the external structure is pressed against the groove. In this way, the external structure may be pressed against a groove at a different location, to adjust a length of the pressurized area, so as to adjust ratios of wrist circumferences of different people to the length of the pressurized area to proper ratios. This improves blood pressure signal collection stability and blood pressure measurement accuracy. In addition, there is no need to design airbags with different lengths for people with different wrist circumferences. This reduces costs of the airbag and a blood pressure measurement apparatus. After a wrist circumference of a user changes, there is no need to replace the airbag and the blood pressure measurement apparatus. This provides good experience and better blood pressure measurement accuracy for the user.

In a possible embodiment, the airbag may be made of an elastic material such as silica gel, polyvinyl chloride, or polyurethane elastomer rubber.

In a possible embodiment, the airbag has a first surface and a second surface that are disposed back-to-back, the second surface is disposed to be in contact with skin, each groove is recessed on the first surface and/or the second surface, and each air hole is disposed on the first surface and/or the second surface.

In a possible embodiment, two air holes are distributed between every two adjacent grooves, one air hole is configured to communicate with an air pump, and the other air hole is configured to communicate with a barometric pressure sensor. Each groove is recessed on the first surface, each air hole is disposed on the first surface, and the two air holes between two adjacent grooves are disposed in parallel along the width direction of the airbag.

In a possible embodiment, the airbag sequentially includes a base section, an adjustable section, and a connection section along the length direction of the airbag, the grooves are distributed on the adjustable section, a groove located at an edge of one side of the adjustable section is a boundary between the adjustable section and the base section, and a groove located at an edge of the other side of the adjustable section is a boundary between the adjustable section and the connection section.

In a possible embodiment, the grooves are distributed at equal intervals along a length direction of the adjustable section.

Alternately, the grooves are distributed along a length direction of the adjustable section in a manner in which the grooves are sparse at two ends and dense in the middle.

In a possible embodiment, a length of the base section is from 120 mm to 140 mm, a length of the adjustable section is from 90 mm to 180 mm, and 3 to 10 grooves are evenly distributed on the adjustable section.

In a possible embodiment, a sealed cavity is disposed at an inner side of the airbag facing the air hole, the blocking part is disposed in the sealed cavity, an outer peripheral wall of the blocking part fits an inner peripheral wall of the sealed cavity, and a spring is connected between the blocking part and the sealed cavity.

In a possible embodiment, an outer peripheral wall of the blocking part and an inner peripheral wall of the sealed cavity are both in a serrated shape and form a concave-convex fit with each other.

In a possible embodiment, the blocking parts are made of hard plastic or hard rubber.

According to a second aspect, an embodiment further provides a blood pressure measurement apparatus, including a cavity, a pressing part, an air pump, a barometric pressure sensor, and an airbag. The airbag has a fixed end and a free end, the fixed end is fixed in the cavity, and the free end can be drawn out of the cavity.

The pressing part is movably disposed in the cavity and can be pressed against the groove to divide the airbag into a pressurized area and a non-pressurized area, where the pressurized area is close to the free end.

The air pump is configured to communicate with the air hole to inflate and pressurize the pressurized area, and the barometric pressure sensor is configured to communicate with the air hole to collect a pressure signal in the pressurized area in real time, to obtain a pulse wave signal. The cavity is configured to accommodate the non-pressurized area of the airbag, and when the airbag does not work, accommodate the entire airbag.

According to the blood pressure measurement apparatus provided in this embodiment, the pressing part, the air pump, and the barometric pressure sensor are disposed. In this way, the pressing part may be pressed against a groove at a different location on the airbag, to divide the airbag into the pressurized area and the non-pressurized area and adjust a length of the pressurized area, so as to adjust ratios of wrist circumferences of different people to the length of the pressurized area to proper ratios. This improves blood pressure signal collection stability and blood pressure measurement accuracy. In addition, the cavity is disposed, so that the airbag can be accommodated in the cavity when the airbag is not used, to prevent the airbag from being damaged. In addition, when the blood pressure measurement apparatus is a blood pressure watch, after the airbag is accommodated in the cavity, the blood pressure watch can be used and comfortably worn as an ordinary watch.

In a possible embodiment, the pressing part is plate-shaped, a first section of the groove is V-shaped, the first section is parallel to a length direction of the airbag, and is perpendicular to a width direction of the airbag, and the pressing part can be pressed against a bottom of the groove from top to bottom, so that the airbag is sealed by pressing at the groove to isolate air flow of the pressurized area from that of the non-pressurized area. The groove is disposed in a V shape, and the V shape has a tip. This ensures that the pressing part can be fully pressed against the bottom of the groove, to ensure sealing of the pressurized area. For an atmospheric pressure range below 300 mm Hg, no airflow flows from the pressurized area to the non-pressurized area.

In a possible embodiment, a rotating shaft is disposed in the cavity, the fixed end is fixed on the rotating shaft, and the rotating shaft is rotatable to wind the airbag around the rotating shaft. The airbag is wound around the rotating shaft, to reduce space occupied by the airbag and the non-pressurized area.

In a possible embodiment, the cavity includes an accommodating part and an extension part connected to each other, an inner diameter of the accommodating part is greater than an inner diameter of the extension part, the rotating shaft is disposed in the accommodating part, the air pump and the barometric pressure sensor are disposed on an outer side of the extension part and penetrate the extension part, and the pressing part is disposed at a joint between the accommodating part and the extension part and functions as a gate of the accommodating part.

In a possible embodiment, in the length direction of the airbag, there is a first distance between a center of the pressing part and a center of a first air nozzle of the air pump and a second distance between a center of the groove and a center of an adjacent air hole, and the first distance is equal to the second distance.

In a possible embodiment, the blood pressure measurement apparatus further includes a fastener, the fastener is disposed on the extension part, the airbag penetrates the fastener, two positioning holes are disposed on a surface of the fastener, and the first air nozzle of the air pump and a second air nozzle of the barometric pressure sensor may respectively pass through the two positioning holes to be inserted into corresponding air holes of the airbag. The fastener is disposed to limit the airbag to some extent. This ensures that the air pump and the barometric pressure sensor are respectively aligned with the two air holes, and facilitates inflation, deflation, extraction, and pressure detection of the airbag. In addition, during movement of the airbag, the air holes and the positioning holes are stuck, to remind a user that the airbag is in a location for fixing.

In a possible embodiment, the blood pressure measurement apparatus further includes a lifting platform configured to lift or lower down the fastener and a corresponding part of the airbag. The fastener and the airbag are lowered down by using the lifting platform, so that the airbag is not limited by the first air nozzle and the second air nozzle when being drawn out. In addition, the fastener and the airbag are lifted by using the lifting platform, and the first air nozzle and the second air nozzle are respectively inserted into corresponding air holes in the lifting process.

In a possible embodiment, the blood pressure measurement apparatus further includes a control board, a first driving part, and a second driving part, the first driving part is configured to drive the rotating shaft to rotate, the second driving part is configured to drive the pressing part to move, and the control board is separately electrically connected to the first driving part, the second driving part, the air pump, the barometric pressure sensor, and the lifting platform.

In a possible embodiment, the blood pressure measurement apparatus further includes a watch core and a watch strap. The cavity, the air pump, the barometric pressure sensor, and the lifting platform are all disposed on the watch core, and the watch strap is disposed on two opposite sides of the watch core.

According to a third aspect, an embodiment further provides a blood pressure measurement method. Human blood pressure is measured by using the blood pressure measurement apparatus, and the method includes the following steps:

drawing out a free end of an airbag and pulling the free end to an outer side of a wrist until the airbag covers a styloid process of ulna;

inserting a first air nozzle of an air pump and a second air nozzle of a barometric pressure sensor into corresponding air holes;

pressing a pressing part against a groove to divide the airbag into a pressurized area and a non-pressurized area;

inflating the pressurized area by using the air pump, and measuring blood pressure by using the barometric pressure sensor;

after the measurement ends, and the air pump is powered off and connected to the atmosphere, deflating the airbag by using the air pump; and

lifting the pressing part and winding the airbag into a cavity.

According to the blood pressure measurement method provided in this embodiment, when the airbag is wound on a wrist, the pressing part is driven to press against the groove, to divide the airbag into the pressurized area and the non-pressurized area. In this way, wrists with different wrist circumferences correspond to pressurized areas with different lengths, and a proper length ratio of a wrist circumference to the pressurized area is ensured, to improve blood pressure measurement accuracy.

### BRIEF DESCRIPTION OF THE DRAWINGS

FIG. 1 is a schematic front view of a blood pressure measurement apparatus according to an embodiment;

FIG. 2 is a schematic top view of a blood pressure measurement apparatus according to an embodiment;

FIG. 3 is a schematic front view of an airbag according to an embodiment;

FIG. 4 is a sectional view of a blocked air hole in FIG. 1;

FIG. 5 is a sectional view of an open air hole in FIG. 1;

FIG. 6 is a schematic top view of an air hole, a sealed cavity, and a blocking part in FIG. 4;

FIG. 7 is a schematic diagram of a circuit of a blood pressure measurement apparatus according to an embodiment; and

FIG. 8 is a schematic of a structure of a blood pressure measurement apparatus when the blood pressure measurement apparatus is a blood pressure watch according to an embodiment.

### DETAILED DESCRIPTION OF THE EMBODIMENTS

To make the problem that needs to be resolved, solutions, and benefits clearer and more comprehensible, the following further describes the accompanying drawings and embodiments. It should be understood that the embodiments described herein are merely used to explain but are not intended as limiting.

It should be noted that when an element is “fastened to” or “disposed on” another element, the element may be directly or indirectly on another element. When an element is “connected to” another element, the element may be directly connected to another element or indirectly connected to another element.

It should be understood that a direction or location relationship indicated by terms such as “length”, “width”, “upper”, “lower”, “front”, “rear”, “left”, “right”, “vertical”, “horizontal”, “top”, “bottom”, “inner”, or “outer” is a direction or a location relationship shown based on the accompanying drawings, is merely used to simplify the descriptions, but is not intended to specify or imply that an indicated apparatus or element needs to have a particular direction, needs to be constructed in a particular direction structure, and needs to be operated in a particular direction, and therefore cannot be construed as a limitation.

In addition, the terms “first” and “second” are merely intended for a purpose of description and shall not be understood as an indication or implication of relative importance or implicit indication of a quantity of indicated features. Therefore, a feature restricted by “first ” or “second” may explicitly indicate or implicitly include one or more such features. In the description, “a plurality of” means two or more than two, unless otherwise limited.

The embodiments relate to an airbag, a blood pressure measurement apparatus, and a blood pressure measurement method. The airbag can be sleeved on a wrist of a human and can be inflated to press radial and ulnar arteries on the wrist of the human, so as to obtain a pulse wave signal. The blood pressure measurement apparatus may be worn on the wrist of the human and can measure blood pressure of the human in real time, to implement dynamic blood pressure monitoring and blood pressure monitoring at night. The blood pressure measurement method is a method for measuring human blood pressure by using the foregoing blood pressure measurement apparatus.

The following briefly describes concepts in the embodiments above.

Blood pressure: Side pressure of blood on a blood vessel wall in a blood vessel. High or low blood pressure affects human health.

Radial artery: A lower part of the radial artery is covered only by the skin and fascia and is a site to palpate the pulse.

Styloid process of ulna: (styloid process of ulna), a Human anatomy term, is a downward tapered prominence on a posteromedial side at the distal end of the ulna, and is palpable on the body surface.

Diastolic pressure: When the human heart relaxes, the arterial blood vessels elastically retract, a pressure generated is called diastolic pressure, also called lower pressure.

Systolic pressure: When the human heart contracts, the pressure in the arteries increases. In the middle of the contraction, the pressure in the arteries is the highest. In this case, the pressure of the blood on the inner wall of the blood vessel is called systolic pressure, also called high pressure.

According to experiments, a length of the airbag is a fixed value, and a coverage range of wrist circumferences of different people is between 130 mm and 220 mm. A span of the wrist circumference is relatively large, and the airbag of a single length cannot cover all people. Experiments show that a ratio of an airbag length to a wrist circumference directly affects stability of collecting a pulse wave signal feature and blood pressure measurement accuracy. If the ratio exceeds 1, that is, when the airbag length is greater than a wrist circumference of a measured person, the airbag inevitably overlaps on a wrist of the measured person. This seriously affects expansion of the airbag and obtaining of a pulse wave signal, and even an effective pulse wave signal cannot be obtained. If the ratio is small, that is, if the airbag length is insufficient to cover an effective part for obtaining the pulse wave signal, peak pressure of the obtained pulse wave signal shifts rightwards, and measured blood pressure is relatively high. In addition, a shift of peak pressure of the pulse wave signal causes instability of a pulse wave signal feature, and further reduces blood pressure measurement accuracy.

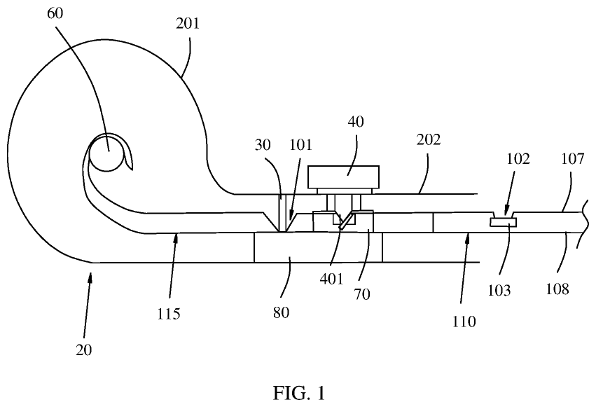

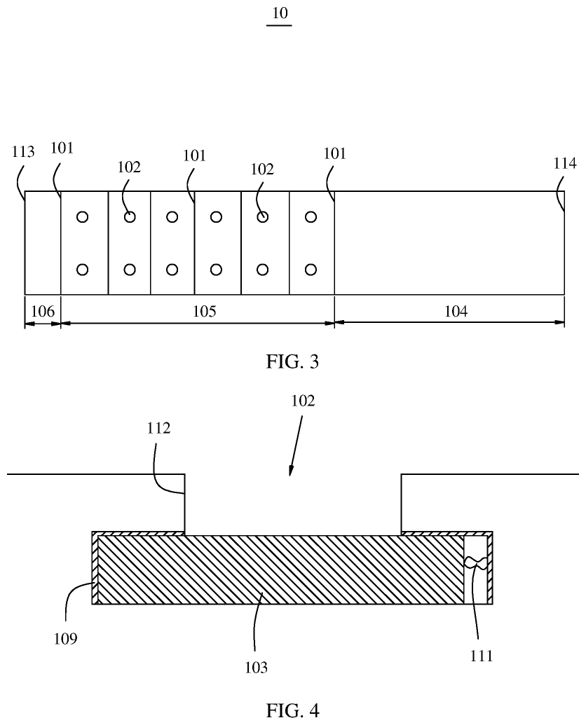

As shown in FIG. 1 to FIG. 3, an airbag 10 provided in the embodiments is described herein. The airbag 10 is used for blood pressure measurement. The airbag 10 is long-strip-shaped, a length extension direction of the airbag 10 is a left-right direction in FIG. 3, and a width direction of the airbag 10 is an up-down direction in FIG. 3. A plurality of grooves 101 are distributed on a surface of the airbag 10 along the length extension direction of the airbag 10, the grooves 101 extend along the width direction of the airbag 10, and each groove 101 is configured to divide the airbag 10 into a pressurized area 110 and a non-pressurized area 115 along the length direction of the airbag 10 when an external structure is pressed against the groove 101. The pressurized area 110 and the non-pressurized area 115 isolate air flow from each other, air holes 102 connecting the inside and the outside of the airbag 10 are distributed between every two adjacent grooves 101 and blocking parts 103 for blocking the air holes 102 are further disposed in the airbag 10.

As shown in FIG. 1, the airbag 10 is formed by sealing an upper surface layer and a lower surface layer. The upper and lower surface layers are stacked together when the airbag 10 is not inflated. When the airbag 10 is inflated, the airbag 10 begins to expand, and the two surface layers are separated from each other. In this case, the upper or lower surface layer abuts against radial and ulnar arteries. Air in the airbag 10 presses the radial and ulnar arteries, so that atmospheric pressure in the airbag 10 is synchronized with a pulse of the radial and ulnar arteries. When a barometric pressure sensor 50 collects a barometric pressure signal in the airbag 10, pulse wave signals of the radial and ulnar arteries can be obtained.

That each groove 101 is configured to divide the airbag 10 into a pressurized area 110 and a non-pressurized area 115 along the length direction of the airbag 10 when an external structure is pressed against the groove 101 means that each of the plurality of grooves 101 may be pressed by the external structure, but only one groove 101 is pressed at a time. When the external structure is pressed against the groove 101, the upper surface layer and the lower surface layer tightly abut against each other at a location of the groove 101. An area on one side of the groove 101 is set as the pressurized area 110, and an area on the other side of the groove 101 is set as the non-pressurized area 115. The left side in FIG. 1 is the non-pressurized area 115, and the right side in FIG. 1 is the pressurized area 110. In other words, the grooves 101 function as scales of the airbag 10. Lengths of the pressurized area 110 and the non-pressurized area 115 change as the external structure is pressed against a groove 101 at a different location. Therefore, the length of the pressurized area 110 is adjustable. Pressurized areas 110 with different lengths may be adjusted based on wrist circumferences of different people, to ensure that a same airbag 10 can satisfy people with different wrist circumferences, and ratios of the length of the pressurized area 110 to wrist circumferences of different people can be adjusted to proper ratios. This improves blood pressure measurement accuracy.

The groove 101 penetrates the airbag 10 along the width direction of the airbag 10. When the external structure is fully pressed against the groove 101 along the width direction of the airbag 10, the pressurized area 110 and the non-pressurized area 115 can be fully isolated at least below an atmospheric pressure difference 300 mm Hg. When the atmospheric pressure is less than 300 mm Hg, air in the pressurized area 110 cannot enter the non-pressurized area 115. This ensures sealing of the pressurized area 110 and improves blood pressure measurement accuracy.

Two air holes 102 are distributed between every two adjacent grooves 101. In this way, when any groove 101 is pressed, the pressurized area 110 has at least two air holes 102. The two air holes 102 are respectively configured to communicate with an air pump 40 and the barometric pressure sensor 50. The air pump 40 is configured to inflate the pressurized area 110, so that the pressurized area 110 can expand to press the radial and ulnar arteries. The barometric pressure sensor 50 is configured to connect to the pressurized area 110 to collect the barometric pressure signal in the pressurized area 110 in real time, to obtain a pulse wave signal and implement blood pressure measurement. Additionally, in another embodiment, when the barometric pressure sensor 50 is integrated into the air pump 40, one air hole 102 may alternatively be distributed between every two adjacent grooves 101.

The blocking part 103 initially blocks the air hole 102, to seal the air hole 102 and prevent air leakage. When a first air nozzle 401 of the air pump 40 or a second air nozzle 501 of the barometric pressure sensor 50 presses against the blocking part 103, the blocking part 103 opens the air hole 102, the air pump 40 may inflate the pressurized area 110 through the air hole 102, and the barometric pressure sensor 50 may collect the barometric pressure signal in the pressurized area 110 through the air hole 102.

According to the airbag 10 provided in this embodiment, the plurality of grooves 101 are distributed in the length direction of the airbag, and each groove 101 is configured to divide the airbag 10 into the pressurized area 110 and the non-pressurized area 115 along the length direction of the groove 101 when the external structure is pressed against the groove 101. In this way, the external structure may be pressed against a groove 101 at a different location, to adjust a length of the pressurized area 110, so as to adjust ratios of wrist circumferences of different people to the length of the pressurized area 110 to proper ratios. This improves blood pressure signal collection stability and blood pressure measurement accuracy. In addition, there is no need to design airbags 10 with different lengths for people with different wrist circumferences. This reduces costs of the airbag 10 and a blood pressure measurement apparatus. When a wrist circumference of a user changes, there is no need to replace the airbag 10 and the blood pressure measurement apparatus. This provides good experience and better blood pressure measurement accuracy for the user.

In an embodiment, the airbag 10 may be made of an elastic material such as silica gel, polyvinyl chloride, or polyurethane elastomer rubber.

As shown in FIG. 1 and FIG. 3, the airbag 10 has a first surface 107 and a second surface 108 that are disposed back-to-back. The second surface 108 is disposed to be in contact with skin, and each groove 101 is recessed on the first surface 107. In this way, when adjusting the length of the pressurized area 110, an external structure that is pressed against the groove 101 may be disposed on a same side of the airbag 10. Different grooves 101 are moved to the external structure for pressing against, to adjust the length of the pressurized area 110 without adjusting a location of the external structure. It can be understood that in another embodiment, all the grooves 101 may be recessed on the second surface 108, or some of the grooves 101 are recessed on the first surface 107 and some of the grooves 101 are recessed on the second surface 108. This is not limited herein.

As shown in FIG. 1 and FIG. 3, two air holes 102 are distributed between every two adjacent grooves 101. One air hole 102 is configured to communicate with the air pump 40, and the other air hole 102 is configured to communicate with the barometric pressure sensor 50. Each air hole 102 is disposed on the first surface 107, and the air holes 102 between every two adjacent grooves 101 are disposed in parallel along the width direction of the airbag 10. Distances between adjacent air holes 102 are the same along the length direction of the airbag 10. The air holes 102 are disposed on a same surface of the airbag 10. In this way, locations of the air pump 40 and the barometric pressure sensor 50 can be fixed. When the length of the pressurized area 110 is adjusted, the air holes 102 are moved to locations corresponding to the air pump 40 and the barometric pressure sensor 50 in sequence, and the air pump 40 and the barometric pressure sensor 50 do not need to be moved. In addition, two air holes 102 between two grooves 101 are disposed in parallel along the width direction. In this way, sizes occupied by the two air holes 102 in the length direction of the airbag 10 can be reduced as much as possible. Therefore, a distance between two adjacent grooves 101 is reduced as much as possible, that is, a distance between adjacent scales is reduced. This improves blood pressure measurement accuracy. It can be understood that in another embodiment, all the air holes 102 may be disposed on the second surface 108, or some of the air holes 102 are disposed on the first surface 107 and some of the air holes 102 are disposed on the second surface 108. This is not limited herein.

As shown in FIG. 3, the airbag 10 sequentially includes a base section 104, an adjustable section 105, and a connection section 106 along the length direction of the airbag 10. The base section 104, the adjustable section 105, and the connection section 106 are sequentially connected from right to left in FIG. 3. The grooves 101 are distributed on the adjustable section 105. A groove 101 located on an edge of one side of the adjustable section 105 is a boundary between the adjustable section 105 and the base section 104, and a groove 101 located on an edge of the other side of the adjustable section 105 is a boundary between the adjustable section 105 and the connection section 106. That is, the groove 101 located on the leftmost side and the groove 101 located on the rightmost side divide the entire airbag 10 into the base section 104, the adjustable section 105, and the connection section 106. One end of the airbag 10 may be fixed by disposing the connection section 106. In addition, because each person has a wrist circumference, even for a person with a thin wrist, the base section 104 may be of a value of a minimum wrist circumference obtained through data survey. No groove 101 is disposed in the base section 104 because the base section 104 is already of the minimum wrist circumference value, and the entire base section 104 needs to be used even if a small wrist circumference is measured. In other words, the base section 104 is disposed to reduce a distribution range of the grooves 101, and a manufacturing process and costs of the grooves 101. This can also reduce a length adjustment range for the user and reduce adjustment difficulty for the user.

As shown in FIG. 3, the grooves 101 are distributed at equal intervals along a length direction of the adjustable section 105. That is, distances between every two adjacent grooves 101 are equal. In this way, each groove 101 functions as a scale on the adjustable section 105. The scales are even, so that users with different wrist circumferences can adjust the length of the pressurized area 110 to a most proper length, and a deviation is not large. This improves blood pressure signal collection stability and blood pressure measurement accuracy. It can be understood that, in another embodiment, the grooves 101 may also be distributed along a length direction of the adjustable section 105 in a manner in which the grooves are sparse at two ends and dense in the middle. Because wrist circumferences of most people are concentrated, and there are fewer people with particularly large or particularly small wrist circumferences, the grooves 101 close to the middle may be distributed densely, and the grooves 101 close to the two ends may be distributed sparsely. In this way, adjustment precision of the adjustable section 105 of people with moderate wrist circumferences may be finer. This further improves blood pressure signal collection stability and blood pressure measurement accuracy.

According to statistics, wrist circumferences of different people range from 130 mm to 220 mm .That is, the minimum wrist circumference is 130 mm, and the maximum wrist circumference is 220 mm. Therefore, in this embodiment, a length of the base section 104 is set to 130 mm, a length of the adjustable section 105 is set to 90 mm, seven grooves 101 are evenly distributed on the adjustable section 105, and a distance between two adjacent grooves 101 is 15 mm. In this way, not only wrist circumference values within a range of 130 mm to 220 mm can be covered, but also a scale exists at every 15 mm between 130 mm and 220 mm. That is, a length adjustment deviation is about 7.5. mm. The length of the airbag 10 in this embodiment is set to be able to wrap around the wrist. If there is a 7.5 mm deviation, it occurs on the back of the wrist, and does not affect measurement accuracy. This ensures measurement accuracy of different wrist circumferences. It can be understood that, in another embodiment, lengths of the base section 104 and the adjustable section 105 may be properly adjusted based on an actual application requirement, and a distance between two adjacent grooves 101 may also be properly adjusted. For example, a length of the base section is any value from 120 mm to 140 mm; a length of the adjustable section 105 is any value from 90 mm to 180 mm; 3, 4, 5, 6, 8, 9, or 10 grooves 101 are evenly distributed on the adjustable section 105. This is not limited herein.

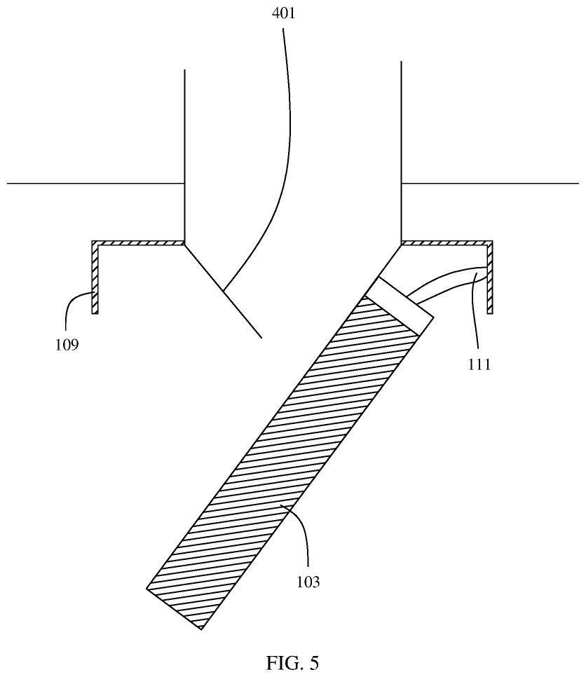

As shown in FIG. 4 and FIG. 5, a sealed cavity 109 is disposed at a location of an inner side of the airbag 10 facing the air hole 102, the sealed cavity 109 communicates with the air hole 102, the sealed cavity 109 is round, the sealed cavity 109 and the air hole 102 are disposed concentrically, and an inner diameter of the sealed cavity 109 is greater than an inner diameter of the air hole 102. The blocking part 103 is disposed in the sealed cavity 109. The blocking part 103 is cylindrical, and an outer peripheral wall of the blocking part 103 fits an inner peripheral wall of the sealed cavity 109, so that there is no gap between the blocking part 103 and the sealed cavity 109. “The outer peripheral wall of the blocking part 103” herein is the outer peripheral wall of the blocking part 103 along a circumferential direction, and “the inner peripheral wall of the sealed cavity 109” is the inner peripheral wall of the sealed cavity 109 along the circumferential direction. A spring 111 is connected between the blocking part 103 and the sealed cavity 109, and one end of the blocking part 103 is connected to the inner peripheral wall of the sealed cavity 109 through the spring 111. When no force is applied to the spring 111, the blocking part 103 is in a closed state, and the blocking part 103 is accommodated in the sealed cavity 109 to block the air hole 102. When the first air nozzle 401 or the second air nozzle 501 is inserted into the corresponding air hole 102, the blocking part 103 in the air hole 102 is pushed away by the first air nozzle 401 or the second air nozzle 501, and a force is applied to the spring 111 connected to the blocking part 103. Because ends of the first air nozzle 401 and the second air nozzle 501 are funnel-shaped, after the first air nozzle 401 pushes away the blocking part 103, the first air nozzle 401 is connected to the airbag 10, and the air pump 40 may inflate and pressurize the airbag 10. When the first air nozzle 401 or the second air nozzle 501 is drawn out of the air hole 102, the blocking part 103 closes the air hole 102 under an effect of a restoring force of the spring 111.

As shown in FIG. 4, the sealed cavity 109 and the air hole 102 are disposed separately along an axial direction of the air hole 102, and the sealed cavity 109 and the air hole 102 are connected over a connection ring 112.

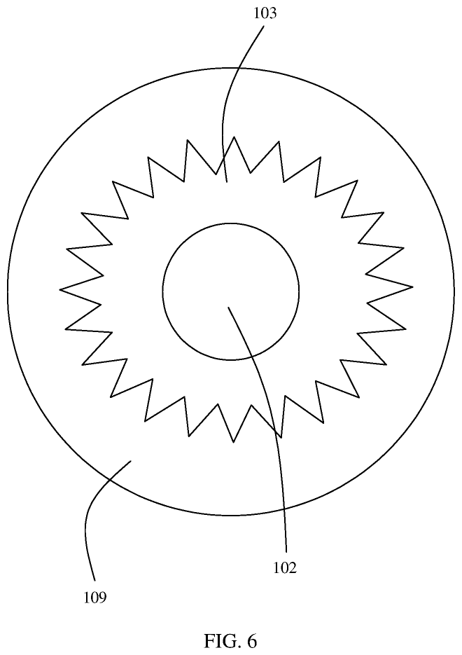

As shown in FIG. 6, both the outer peripheral wall of the blocking part 103 and the inner peripheral wall of the sealed cavity 109 are in a serrated shape and form a concave-convex fit with each other. This ensures connection tightness between the blocking part 103 and the sealed cavity 109, to improve air tightness of the airbag 10 and blood pressure measurement accuracy.

The blocking part 103 may be made of hard plastic or hard rubber. In this way, when the airbag 10 is inflated and expands under pressure, the blocking part 103 does not deform. This can ensure that air in the airbag 10 does not leak out from the corresponding air hole 102, to ensure air tightness of the airbag 10.

Based on a same application concept, an embodiment further provides a blood pressure measurement apparatus. As shown in FIG. 1 to FIG. 3, the blood pressure measurement apparatus includes a cavity 20, a pressing part 30, the air pump 40, the barometric pressure sensor 50, and the airbag 10. The airbag 10 has a fixed end 113 and a free end 114. The fixed end 113 is fixed in the cavity 20, and the free end 114 extends from the cavity 20 and may be fixed on the back of the wrist after wrapping around the wrist. The pressing part 30 is movably disposed in the cavity 20 and can be pressed against the groove 101 to divide the airbag 10 into the pressurized area 110 and the non-pressurized area 115. The pressurized area 110 is close to the free end 114, the non-pressurized area 115 is close to the fixed end 113, and the non-pressurized area 115 is accommodated in the cavity 20. The pressurized area 110 may extend from the cavity 20 and may be wound around the wrist to press human radial and ulnar arteries. The air pump 40 is disposed outside the cavity 20 and is configured to communicate with an air hole 102 to inflate the pressurized area 110. The barometric pressure sensor 50 is disposed outside the cavity 20 and is configured to communicate with an air hole 102 to collect a pressure signal in the pressurized area 110 in real time to obtain a pulse wave signal. Two air holes 102 are disposed between two adjacent grooves 101, and the two air holes 102 are configured to respectively communicate with the air pump 40 and the barometric pressure sensor 50. The cavity 20 is configured to accommodate the non-pressurized area 115 of the airbag 10, and when the airbag 10 does not work, accommodate the entire airbag 10.

In use, the cavity 20 is first fixed on the back of the wrist, then the free end 114 of the airbag 10 is drawn out of the cavity 20, and the free end 114 is pulled from the back of the wrist to pass one side, the front, and the other side of the wrist in sequence, so that the airbag 10 wraps around the wrist, and the free end 114 is fixed on the back of the wrist from the other side. Then, the pressing part 30 is driven to press against a corresponding groove 101 on the back of the wrist, to divide the airbag 10 into the pressurized area 110 and the non-pressurized area 115. The pressing part 30 is set at a location, so that the pressurized area 110 can completely cover the front and the two sides of the wrist. Finally, the air pump 40 inflates the pressurized area 110, and the barometric pressure sensor 50 collects a pressure signal in the pressurized area 110 in real time to obtain a pulse wave signal. When the blood pressure measurement apparatus is a blood pressure watch, because the cavity 20 is disposed on a watch core, when the blood pressure watch is worn on the wrist, the cavity 20 is fixed on the back of the wrist, and the free end 114 may also be fixed by pressing by a watch strap; or a fastener such as a hook may be disposed on the free end 114 to fix the free end on the watch core.

According to the blood pressure measurement apparatus provided in this embodiment, the pressing part 30, the air pump 40, and the barometric pressure sensor 50 are disposed. In this way, the pressing part 30 may be pressed against a groove 101 at a different location on the airbag 10, to divide the airbag 10 into the pressurized area 110 and the non-pressurized area 115 and adjust a length of the pressurized area 110, so as to adjust ratios of wrist circumferences of different people to the length of the pressurized area 110 to proper ratios for measurement. This improves blood pressure signal collection stability and blood pressure measurement accuracy. In addition, the cavity 20 is disposed, so that the airbag 10 can be accommodated in the cavity 20 when the airbag 10 is not used, to prevent the airbag 10 from being damaged. In addition, when the blood pressure measurement apparatus is a blood pressure watch, after the airbag 10 is accommodated in the cavity 20, the blood pressure watch can be used and comfortably worn as an ordinary watch.

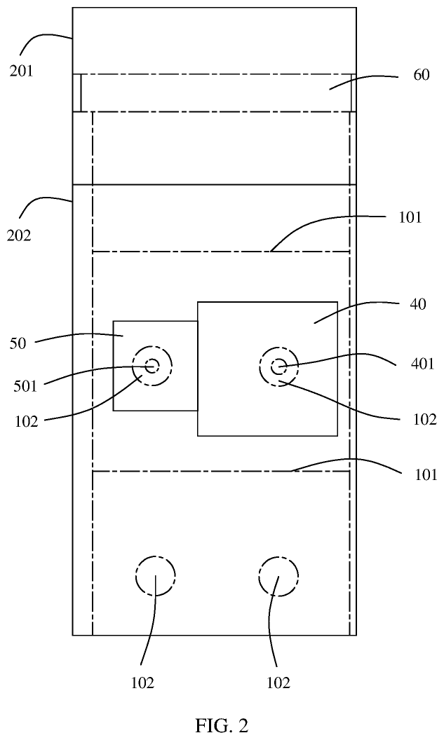

As shown in FIG. 1 and FIG. 2, a rotating shaft 60 is disposed in the cavity 20, the fixed end 113 is fixed on the rotating shaft 60, and the rotating shaft 60 may rotate to wind the airbag 10 around the rotating shaft 60. The rotating shaft 60 is connected to a first driving part 100. The first driving part 100 is disposed in the cavity 20 or disposed outside the cavity 20. The first driving part 100 can drive the rotating shaft 60 to rotate, and the first driving part 100 is electrically connected to a control board 90 in the blood pressure measurement apparatus. The first driving part 100 is a stepper motor. In actual application, when the measurement ends and the airbag 10 is not needed, the control board 90 sends a control command to the first driving part 100, and the first driving part 100 drives the rotating shaft 60 to rotate in steps, to wind the airbag 10 around the rotating shaft 60 and accommodate the airbag 10 in the cavity 20. When the airbag 10 is needed, the fixed end 113 of the airbag 10 is manually pulled, and a part of the airbag 10 is unwound from the rotating shaft 60.

As shown in FIG. 1 and FIG. 2, the cavity 20 includes an accommodating part 201 and an extension part 202, and the accommodating part 201 and the extension part 202 are connected to each other along the length direction of the airbag 10. The accommodating part 201 is approximately cylindrical, an inner diameter of the accommodating part 201 is greater than an inner diameter of the extension part 202, and the rotating shaft 60 is disposed in the accommodating part 201. That is, the accommodating part 201 is configured to accommodate the airbag 10, and the inner diameter of the accommodating part 201 is set to be larger to accommodate the airbag 10. The air pump 40 and the barometric pressure sensor 50 are disposed on an outer side of the extension part 202 and penetrate the extension part 202, and the first air nozzle 401 and the second air nozzle 501 extend into the extension part 202. In this way, the air pump 40 and the barometric pressure sensor 50 may be connected to the airbag 10 through the air holes 102 located in the extension part 202. The pressing part 30 is disposed at a joint between the accommodating part 201 and the extension part 202 and functions as a gate of the accommodating part 201. That is, the pressing part 30 can be pressed against only a groove 101 at the joint between the accommodating part 201 and the extension part 202. In other words, the non-pressurized area 115 is located in the accommodating part 201, and the pressurized area 110 extends from the extension part 202.

As shown in FIG. 1, there is a first distance between a center of the pressing part 30 and a center of the first air nozzle 401 of the air pump 40 and a second distance between a center of the groove 101 and a center of an adjacent air hole 102, and the first distance is equal to the second distance. After locations of the pressing part 30, the air pump 40, and the barometric pressure sensor 50 are fixed, as long as the pressing part 30 is pressed against one of the grooves 101, there are two air holes 102 facing the air pump 40 and the sensor, and the air nozzles only need to be inserted into the air holes 102.

As shown in FIG. 1 and FIG. 2, the pressing part 30 is plate-shaped, and the pressing part 30 is vertically disposed. The pressing part 30 extends along the width direction of the airbag 10, and a width of the pressing part 30 in the width direction of the airbag 10 is greater than or equal to the width of the airbag 10. In this way, the pressing part 30 can be fully pressed against the entire groove 101, to ensure sealing of the pressurized area 110. A first section of the groove 101 is V-shaped, the first section is parallel to the length direction of the airbag 10, and is perpendicular to the width direction of the airbag 10, and the pressing part 30 may be pressed against a bottom of the groove 101 from top to bottom, so that the airbag 10 is sealed by pressing at the groove 101, to isolate the air flow of the pressurized area 110 from that of the non-pressurized area 115. When the pressing part 30 is pressed against the bottom of the groove 101 from top to bottom, the upper and lower surface layers of the airbag 10 abut against each other at the groove 101, so that the entire airbag 10 is divided into the pressurized area 110 and the non-pressurized area 115. In this embodiment, the groove 101 is disposed in a V shape, and the V shape has a tip. This ensures that the pressing part 30 can be fully pressed against the bottom of the groove 101, to ensure sealing of the pressurized area 110.

The pressing part 30 is connected to a second driving part 120, and the second driving part 120 is configured to drive the pressing part 30 to move from top to bottom, so that the pressing part 30 is pressed against the groove 101. In addition, after the airbag 10 is accommodated in the accommodating part 201, the accommodating part 201 may be further isolated from the outside, to prevent the airbag 10 from getting out of the accommodating part 201.

As shown in FIG. 1, the blood pressure measurement apparatus further includes a fastener 70. The fastener 70 is disposed in the extension part 202, the airbag 10 penetrates the fastener 70, and the fastener 70 has a circumferential limiting function on the airbag 10. In other words, when the airbag 10 penetrates the fastener 70, the airbag 10 can move only along the length direction of the airbag 10. Two positioning holes (not shown in the figure) are disposed on a surface of the fastener 70. The two positioning holes are disposed in parallel along the width direction of the airbag 10, and a distance between centers of the two positioning holes is equal to a distance between centers of the two air holes 102. That is, when the airbag 10 moves, two air holes 102 are disposed facing the two positioning holes each time. Because the positioning holes and the air holes 102 are disposed, and a protrusion is disposed on an inner side of the fastener 70, when the air holes 102 pass the positioning holes, the air holes 102 and the positioning holes are stuck when the air holes 102 are in contact with the positioning holes because the protrusion blocks the airbag 10, to remind that the air holes 102 are aligned with the positioning holes. In this case, the pressing part 30 may be driven downward to press against the groove 101, and the first air nozzle 401 of the air pump 40 and the second air nozzle 501 of the barometric pressure sensor 50 may be driven to respectively pass through the two positioning holes to respectively insert into the two corresponding air holes 102 of the airbag 10. In this embodiment, the fastener 70 is disposed, so that the airbag 10 has a limiting and positioning function. This ensures that the air pump 40 and the barometric pressure sensor 50 are respectively aligned with the two air holes 102, and facilitates inflation, deflation, extraction, and pressure detection of the airbag 10.

As shown in FIG. 1, the blood pressure measurement apparatus further includes a lifting platform 80, and the lifting platform 80 is configured to lift or lower down the fastener 70 and a corresponding part of the airbag 10. When not working, the lifting platform 80 is disposed outside a bottom of the extension part 202, and the fastener 70 is disposed on the lifting platform 80 and is located in the extension part 202. In this case, both the fastener 70 and the corresponding part of the airbag 10 are located at the bottom of the extension part 202. This facilitates drawing and winding of the airbag 10 and prevents the airbag 10 from interfering with the first air nozzle 401 and the second air nozzle 501. When the airbag 10 has been drawn out for a required length, the fastener 70 and the corresponding part of the airbag 10 may be lifted to an approximate middle height of the extension part 202 by using the lifting platform 80. In a process of lifting the airbag 10, the first air nozzle 401 and the second air nozzle 501 are respectively inserted into the corresponding air holes 102.

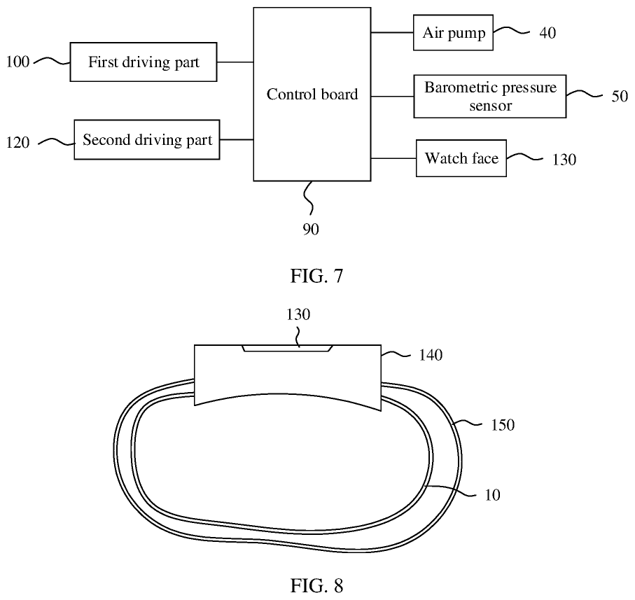

As shown in FIG. 7, the blood pressure measurement apparatus further includes the control board 90. The control board 90 is electrically connected to the first driving part 100, the second driving part 120, the air pump 40, the barometric pressure sensor 50, and the lifting platform 80. In this way, working sequences of the first driving part 100, the second driving part 120, the air pump 40, the barometric pressure sensor 50, and the lifting platform 80 can be separately controlled by the control board 90.

As shown in FIG. 8, the blood pressure measurement apparatus is a blood pressure watch, and further includes a watch core 140 and a watch strap 150. The cavity 20, the air pump 40, the barometric pressure sensor 50, and the lifting platform 80 are all disposed on the watch core 140. A watch face 130 is disposed on the watch core 140, and the watch face 130 is electrically connected to the control board 90. A blood pressure value measured by the barometric pressure sensor 50 may be displayed on the watch face 130, and the watch strap 150 is disposed on two opposite sides of the watch core 140.

Based on a same application concept, an embodiment further provides a blood pressure measurement method. Blood pressure measurement is performed by using the foregoing blood pressure measurement apparatus, and the method includes the following steps.

The gate of the accommodating part 201 is opened.

When the airbag 10 is not used, the entire airbag 10 is wound around the fastener 70, the free end 114 of the airbag 10 stays on the fastener 70, the gate in the accommodating part 201 is closed by using the pressing part 30, and the pressing part 30 presses the free end 114 of the airbag 10, so that the free end 114 of the airbag 10 is fixed on the fastener 70. When the user wants to measure blood pressure, the pressing part 30 needs to be lifted, so that the user draws the airbag 10 out of the accommodating part 201 by hand.

The free end 114 of the airbag 10 is drawn out, and the free end 114 is pulled based on the wrist circumference until the airbag covers the styloid process of ulna on the outer side of the wrist. A hook may be disposed at the free end 114 to fix the free end 114 on the watch core 140, or the free end 114 may be fixed by pressing the free end 114 by the watch strap 150.

Based on an experimental conclusion of a ratio of the length of the airbag 10 to the user's measured wrist circumference, an optimal ratio is between 0.8 and 0.85. That is, the airbag 10 needs to cover the front and the two sides of the wrist. In addition, a start location of the pressurized area 110 is located on the back of the wrist (the gate of the cavity 20 is a boundary between the pressurized area 110 and the non-pressurized area 115, and the gate is located in the watch core on the back of the wrist). Therefore, the tail of the pressurized area 110 of the airbag 10 should reach the styloid process of ulna of the wrist of the user. In this way, the pressurized area 110 of the airbag 10 can cover the front and the two sides of the wrist, so that a ratio of the length the pressurized area 110 to a wrist circumference reaches a proper ratio range. This ensures accuracy and stability of a pulse wave signal obtained by the barometric pressure sensor 50 during blood pressure measurement, to ensure blood pressure measurement accuracy.

The lifting platform 80 is driven to lift, so that the first air nozzle 401 of the air pump 40 and the second air nozzle 501 of the barometric pressure sensor 50 are inserted into corresponding air holes 102.

In this embodiment, the first surface 107 (a front surface) of the airbag 10 has parallel air holes 102 with six gears. When the user draws the airbag 10 out of the accommodating part 201 by hand and pull the free end to the styloid process of ulna on the outer side of the wrist, the airbag 10 passes through the fastener 70, and the air holes 102 on the upper surface of the airbag 10 are stuck, to remind the user that the airbag 10 is pulled to a location for fixing. In this case, the airbag 10 has been pulled out and fixed, and in a process in which the lifting platform 80 lifts, the first air nozzle 401 and the second air nozzle 501 are respectively inserted into the corresponding air holes 102.

The pressurized area 110 is inflated by using the air pump 40, and blood pressure is measured by using the barometric pressure sensor 50.

According to the foregoing steps, the airbag 10 is pulled to an optimal length, the air nozzles of the air pump 40 and the barometric pressure sensor 50 are inserted into the corresponding air holes 102 of the airbag 10, and preparation works before blood pressure measurement are completed. In this case, the air pump 40 can inflate and pressurize the airbag 10, to start a blood pressure measurement process. The air pump 40 inflates the airbag 10, to increase pressure inside the airbag 10, and the airbag 10 expands, and presses the radial and ulnar arteries at the wrist. The barometric pressure sensor 50 collects a pressure signal in the airbag 10 in real time. Because the radial and ulnar arteries are pressed, a pulse wave signal is superimposed on the signal collected by the barometric pressure sensor 50. The pulse wave signal is obtained from the original signal, features (including a peak pressure, a maximum slope point, and the like) of the pulse wave signal are calculated, and final diastolic pressure and systolic pressure values are calculated by using a blood pressure model.

The measurement ends, the air pump 40 is powered off and connected to the atmosphere, and the airbag 10 is deflated by using the air pump 40.

The air pump 40 not only has a function of inflating the airbag 10, but also has a function of extracting air from the airbag 10. When the blood pressure measurement ends, the air pump 40 deflates the pressurized area 110 of the airbag 10. However, after the deflation, some air remains in the pressurized area 110 of the airbag 10. If this air is not extracted, in a process of winding the airbag 10 into the accommodating part 201, because the air nozzles of the air pump 40 and the barometric pressure sensor 50 are drawn out of the air holes 102, and the airbag 10 is sealed, the airbag 10 cannot be wound into the accommodating part 201. Therefore, before winding, the air pump 40 needs to extract the air remaining in the pressurized area 110 of the airbag 10. After the air is extracted, the air nozzles of the air pump 40 and the barometric pressure sensor 50 are drawn out of the air holes 102.

The pressing part 30 is lifted, the lifting platform 80 is driven to lower down, and the rotating shaft 60 is started to rotate to wind the airbag 10 into the cavity 20.

It can be understood that driving action instructions in the foregoing steps are all sent by the control board 90 and completed by the foregoing corresponding parts.

Finally, it should be noted that the foregoing descriptions are merely implementations of embodiments, but are not intended to limit the scope of the embodiments. Any variation or replacement shall fall within the scope of the embodiments.

1. An airbag, configured to measure blood pressure, wherein the airbag is long-strip-shaped, the airbag comprising:
a plurality of grooves distributed on a surface of the airbag along a length extension direction of the airbag, wherein the plurality of grooves extends along a width direction of the airbag, each groove of the plurality of grooves is configured to divide the airbag into a pressurized area and a non-pressurized area along the length direction of the airbag when an external structure is pressed against the groove, the pressurized area and the non-pressurized area isolate air flow from each other;
air holes distributed between every two adjacent grooves, wherein the air holes are used to connect the inside and the outside of the airbag; and
blocking parts for blocking the air holes.

2. The airbag according to claim 1, wherein the airbag has a first surface and a second surface that are disposed back-to-back, the second surface is configured to be in contact with skin, each groove is disposed on the first surface, and each air hole is disposed on the first surface.

3. The airbag according to claim 2, wherein two air holes are distributed between every two adjacent grooves, one air hole is configured to be connected with an air pump, the other air hole is configured to be connected with a barometric pressure sensor, each groove is disposed on the first surface, each air hole is disposed on the first surface, and the two air holes between two adjacent grooves are disposed in parallel along the width direction of the airbag.

4. The airbag according to claim 1, wherein the airbag sequentially comprises a base section, an adjustable section, and a connection section along the length direction of the airbag, the grooves are distributed on the adjustable section, a groove located at an edge of one side of the adjustable section is a boundary between the adjustable section and the base section, and a groove located at an edge of the other side of the adjustable section is a boundary between the adjustable section and the connection section.

5. The airbag according to claim 4, wherein the grooves are distributed at equal intervals along a length direction of the adjustable section; or the grooves are distributed along a length direction of the adjustable section in a manner in which the grooves are sparse at two ends and dense in the middle.

6. The airbag according to claim 4, wherein a length of the base section is from 120 mm to 140 mm, a length of the adjustable section is from 90 mm to 180 mm, and 3 to 10 grooves are evenly distributed on the adjustable section.

7. The airbag according to claim 1, wherein a sealed cavity is disposed at an inner side of the airbag facing the air hole, the blocking part is disposed in the sealed cavity, an outer peripheral wall of the blocking part fits an inner peripheral wall of the sealed cavity, and a spring is configured to be connected between the blocking part and the sealed cavity.

8. The airbag according to claim 7, wherein an outer peripheral wall of the blocking part and an inner peripheral wall of the sealed cavity are both in a serrated shape and form a concave-convex fit with each other.

9. The airbag according to claim 7, wherein the blocking parts is made of hard plastic or hard rubber.

10. The blood pressure measurement apparatus according to claim 20, wherein the airbag has a fixed end and a free end, the fixed end is fixed in the cavity, the free end can be drawn out of the cavity, the pressing part is movably disposed in the cavity and can be pressed against the groove to divide the airbag into a pressurized area and a non-pressurized area, the pressurized area is close to the free end, the air pump is configured to be connected to the air hole to inflate and pressurize the pressurized area, and the barometric pressure sensor is configured to be connected to the air hole to collect a pressure signal in the pressurized area in real time, to obtain a pulse wave signal.

11. The blood pressure measurement apparatus according to claim 10, wherein the pressing part is plate-shaped, a first section of the groove is V-shaped, the first section is parallel to a length direction of the airbag, and is perpendicular to a width direction of the airbag, and the pressing part can be pressed against a bottom of the groove from top to bottom, so that the airbag is sealed by pressing at the groove to isolate air flow of the pressurized area from that of the non-pressurized area.

12. The blood pressure measurement apparatus according to claim 10, wherein a rotating shaft is disposed in the cavity, the fixed end is fixed on the rotating shaft, and the rotating shaft is rotatable to wind the airbag around the rotating shaft.

13. The blood pressure measurement apparatus according to claim 12, wherein the cavity comprises an accommodating part and an extension part configured to be connected to each other, an inner diameter of the accommodating part is greater than an inner diameter of the extension part, the rotating shaft is disposed in the accommodating part, the air pump and the barometric pressure sensor are disposed on an outer side of the extension part and penetrate the extension part, and the pressing part is disposed at a joint between the accommodating part and the extension part and functions as a gate of the accommodating part.

14. The blood pressure measurement apparatus according to claim 13, wherein in the length direction of the airbag, there is a first distance between a center of the pressing part and a center of a first air nozzle of the air pump and a second distance between a center of the groove and a center of an adjacent air hole, and the first distance is equal to the second distance.

15. The blood pressure measurement apparatus according to claim 14, further comprising:
a fastener, the fastener is disposed on the extension part, the airbag penetrates the fastener, two positioning holes are disposed on a surface of the fastener, and the first air nozzle of the air pump and a second air nozzle of the barometric pressure sensor may respectively pass through the two positioning holes to be inserted into corresponding air holes of the airbag.

16. The blood pressure measurement apparatus according to claim 15, further comprising:
a lifting platform configured to lift or lower down the fastener and a corresponding part of the airbag.

17. The blood pressure measurement apparatus according to claim 16, wherein the blood pressure measurement apparatus further comprising:
a control board;
a first driving part, wherein the first driving part is configured to drive the rotating shaft to rotate; and
a second driving part, wherein the second driving part is configured to drive the pressing part to move, and the control board is configured to be separately electrically connected to the first driving part, the second driving part, the air pump, the barometric pressure sensor, and the lifting platform.

18. The blood pressure measurement apparatus according to claim 17, further comprising:
a watch core; and
a watch strap, wherein the cavity, the air pump, the barometric pressure sensor, and the lifting platform are all disposed on the watch core, and the watch strap is disposed on two opposite sides of the watch core.

19. (canceled)

20. A blood pressure measurement apparatus, comprising a cavity, a pressing part, an air pump, a barometric pressure sensor, and the airbag, wherein the airbag comprises:
a plurality of grooves distributed on a surface of the airbag along a length extension direction of the airbag, wherein the plurality of grooves extends along a width direction of the airbag, each groove of the plurality of grooves is configured to divide the airbag into a pressurized area and a non-pressurized area along the length direction of the airbag when an external structure is pressed against the groove, the pressurized area and the non-pressurized area isolate air flow from each other;
air holes distributed between every two adjacent grooves, wherein the air holes are used to connect the inside and the outside of the airbag;
blocking parts for blocking the air holes.

21. The blood pressure measurement apparatus according to claim 20, wherein the airbag has a first surface and a second surface that are disposed back-to-back, the second surface is configured to be in contact with skin, each groove is disposed on the first surface, and each air hole is disposed on the first surface.
